(19) United States
(12) Patent Application Publication
(10) Pub. No.: US 2016/0136063 A1
(43) Pub. Date: May 19, 2016

(54) LONG-WEAR COSMETIC COMPOSITION

(71) Applicant: L'OREAL, Paris (FR)

(72) Inventor: Rui NIIMI, Fujisawa-shi (JP)

(21) Appl. No.: 14/902,889

(22) PCT Filed: Jul. 8, 2014
(86) PCT No.: PCT/JP2014/068650

§ 371 Date: Jan. 5, 2016

(30) Foreign Application Priority Data
Jul. 9, 2013 (JP) 2013-143328

(51) Int. Cl. A61K 8/19; A61K 8/81; A61K 8/365; A61Q 1/02; A61K 8/362

(52) U.S. Cl. CPC A61K 8/19; A61Q 1/02; A61K 8/362; A61K 8/365; A61K 8/8164; A61K 2800/592

(57) ABSTRACT

The present invention relates to a cosmetic composition comprising: (i) at least one oxide of an alkaline earth metal, (ii) at least one carboxylic acid having a pKa equal to or greater than 3.5, and (iii) an aqueous phase. The cosmetic composition according to the present invention can provide a long-wear effect, especially a long-wear makeup effect.

### TECHNICAL FIELD

The present invention relates to a cosmetic composition for the skin. Particularly, the present invention relates to a cosmetic composition having a long-wear property.

### BACKGROUND ART

Sebum secreted from the skin affords an emollient effect to the stratum corneum of the skin prevents intrusion of toxic substances or bacteria from the outside, and controls the release of substances, such as water, out of the body. However, excess sebum secretion has a demerit in that it may cause the makeup to come off. It leads to some phenomena, e.g., a “shiny” or “drab” appearance of the skin, or “unevenness”, “rumpling”, or “disappearance” of the makeup itself, and the like, caused by chronological changes in the cosmetic film formed on the skin.

A variety of investigations have already been conducted from the viewpoint of improving the long-wear property of makeup. For example, should highly moisture-absorbent or oil-absorbent substances, such as porous silica, calcium carbonate, magnesium carbonate, and crystalline cellulose, be mixed into cosmetics, the moisture and sebum components on the skin become adsorbed, which leads to a shortage of skin emollient components and causes a dry skin feeling, and a feeling of skin tightness or skin itchiness. This phenomenon is most likely to occur with persons with dry skin and normal skin, in particular with persons living in an environment where sweat or sebum is secreted less (e.g., persons working in an office). When used on oily skin, these substances have demerits which are apt to present luster due to excess sebum or oily components contained in the cosmetics, thus giving rise to a “shiny” look in the makeup finish.

Under these circumstances, there is a demand for a suitable skin-friendly cosmetic composition which improves the long-wear property of makeup, and can overcome the demerits of the above prior substances.

In view of the above, among the problems to be solved by the present invention is to provide a cosmetic composition which does not obstruct the skin physiology, has a good feeling on the skin, and has a superior long-wear effect for makeup.

Some patent publications describe technology of sebum gelification with metal oxides. For example, JP-B-4961082 discloses a silicone surface treatment with zinc oxide. JP-A-2002-20652 discloses composite particles of and spherical particles coated by silicone-treated zinc oxide. JP-B-3822782 teaches a combination of zinc oxide and hydroxyapatite in a cosmetically acceptable base. JP-B-3702072 discloses a composite where microparticle zinc oxide adheres to a silica surface. JP-B-3073890 discloses a silicone surface treatment with zinc oxide. JP-A-2011-51913 teaches a combination of microparticle titanium dioxide, and at least one chosen from magnesium oxide, calcium oxide, magnesium hydroxide, or calcium hydroxide, and clay. JP-A-2007-277191 teaches that citric acid stabilizes zinc oxide in w/o emulsion cosmetics, though it is silent on the sebum solidification effect.

### DISCLOSURE OF INVENTION

One of the objectives of the present invention is to provide a cosmetic composition which can provide a long-lasting effect, in particular a long-lasting makeup effect.

The above objective of the present invention can be achieved by a cosmetic composition comprising:

(i) at least one oxide of an alkaline earth metal,

(ii) at least one compound having at least one carboxylic acid moiety having a pKa value equal to or greater than 3.5, and

(iii) an aqueous phase.

In the present invention, the pKa value corresponds to −log Ka, Ka being the equilibrium constant for the association of the weak acid, or the acidity constant.

In one preferred embodiment, the weight ratio of the (i) oxide of an alkaline earth metal to the (ii) compound having at least one carboxylic acid moiety may be equal to or less than 1.0.

In one embodiment, the (i) oxide of an alkaline earth metal is an oxide of magnesium or calcium, preferably magnesium oxide.

In one embodiment, the (ii) compound having at least one carboxylic acid moiety may be a non-polymeric compound having a molecular weight greater than 150, preferably greater than 180.

In one embodiment, the non-polymeric compound may be selected from the group consisting of monocarboxylic acids, hydroxycarboxylic acids, dicarboxylic acids, tricarboxylic acids, and mixtures thereof, preferably hydroxycarboxylic acids, dicarboxylic acids, and mixture thereof.

In one embodiment, the non-polymeric compound may be selected from the group consisting of formic acid, glycolic acid, lactic acid, succinic acid, benzoic acid, glutaric acid, adipic acid, azelaic acid, and acetic acid.

In one embodiment, the (ii) compound having at least one carboxylic acid moiety is an anionic polymer compound derived from a carboxylic acid, having a molecular weight greater than 1000.

In one embodiment, the anionic polymer compound derived from a carboxylic acid, is a polymer derived from maleic, fumaric, and itaconic acid.

In one embodiment, the amount of the (i) oxide of an alkaline earth metal is from 0.2% to 5.0%, preferably from 0.3% to 3.0%, and more preferably from 0.5% to 2.0% by weight in relation to the total weight of the cosmetic composition.

In one embodiment, the cosmetic composition according to the present invention may be an emulsion, lotion, gel, or cream.

In one embodiment, the cosmetic composition according to the present invention may be a liquid foundation.

In one embodiment, the present invention relates to use of a combination of (i) at least one oxide of an alkaline earth metal and (ii) at least one compound having at least one carboxylic acid moiety as an agent for improving a long-wear effect, especially a long-wear makeup effect.

In one embodiment, the present invention relates to a cosmetic process comprising applying to keratinous materials comprising human skin, a cosmetic composition as described hereinbefore. The process is preferably for a long-wear effect, especially for a long-wear makeup effect.

The cosmetic composition according to the present invention provides an improved rapid sebum solidification and/or gelification effect. As a result, the cosmetic composition according to the present invention maintains a long-wear effect.

### BEST MODE FOR CARRYING OUT THE INVENTION

After diligent research, the inventors have discovered that it is possible to provide a cosmetic composition which can provide a long-wear effect, especially a long-wear makeup effect.

The cosmetic composition according to the present invention can provide sebum-solidification effects. Therefore, for example, the cosmetic effect is maintained for a long time period.

In the present invention, “sebum solidification” refers to a state in which sebum has been turned into a solid or gel, which can be restated as “sebum gelification”. The time to solidify and/or gelify may not be specifically limited, but it is preferable to achieve sebum solidification and/or gelification within 1 hour, preferably within 30 minutes, and more preferably within 20 minutes.

In one embodiment, the weight ratio of the (i) oxide of an alkaline earth metal to the (ii) compound having at least one carboxylic acid moiety may be from less than 1.0, preferably from 0.05 to 0.50, and more preferably from 0.20 to 0.35.

(i) Oxide of an Alkaline Earth Metal

The cosmetic composition according to the present invention contains (i) at least one oxide of an alkaline earth metal. A single type of oxide or a combination of different types of oxides may be used.

The oxide of an alkaline earth metal is not limited specifically, and those which react with oleic acid, which is regarded as a main component of sebum, may be preferable. The alkaline earth metal constituting the oxide of an alkaline earth metal may be selected from the group consisting of magnesium and calcium, and preferably magnesium.

In a preferred embodiment, the oxide of an alkaline earth metal is in its classical form and not complexed with a substrate such as a pigment and/or a filler.

The oxide of an alkaline earth metal can be surface treated in a conventional manner.

In one embodiment, the oxide of an alkaline earth metal is not surface treated.

In one embodiment, the oxide of an alkaline earth metal may be pre-coated with a coating material such as a silicone compound, a fatty acid, a metal soap, a fluorine-based compound, a silane-coupling agent, and the like. A silicone compound is preferable. The percentage of the coating material in relation to the oxide of an alkaline earth metal may be from 0.1% to 10.0%, preferably from 0.3% to 8.0%, and more preferably from 0.5% to 7.0%.

The oxide of an alkaline earth metal is not limited, but preferably has an average particle size from 0.1 μm to 50 μm, more preferably from 0.5 μm to 30 μm, and even more preferably from 1 μm to 10 μm.

In one embodiment, the amount of the (i) oxide of an alkaline earth metal may be from 0.2% to 5.0%, preferably from 0.3% to 3.0%, and more preferably from 0.5% to 2.0% by weight in relation to the total weight of the cosmetic composition.

(ii) Compound Having at Least One Carboxylic Acid Moiety

The cosmetic composition according to the present invention contains (ii) at least one compound having at least one carboxylic acid moiety. A single type of compound having at least one carboxylic acid moiety or a combination of different types thereof may be used. The compound having at least one carboxylic acid moiety may be a non-polymeric compound or a polymeric compound. The compound having at least one carboxylic acid moiety may be linear, branched, or cyclic. The compound having at least one carboxylic acid moiety may be saturated or unsaturated.

In one embodiment, the compound having a carboxylic acid moiety is a non-polymeric compound having at least one carboxylic acid moiety, preferably having a molecular weight higher than 150, and more preferably higher than 180.

Non-Polymeric Compound Having at Least One Carboxylic Acid Moiety

In one embodiment of the present invention, the compound having at least one carboxylic acid moiety is a non-polymeric compound, which is preferably a weak acid.

Such a non-polymeric compound having at least one carboxylic acid moiety may be chosen from monocarboxylic acids, hydrocarboxylic acids, dicarboxylic acids, tricarboxylic acids, and their mixtures, preferably chosen from hydrocarboxylic acids, dicarboxylic acids, and their mixtures.

Monocarboxylic Acid

Suitable weak monocarboxylic acids for use in the present invention are generally those having a pKa of equal to or greater than 3.5, preferably from 3.5 to 8, and more preferably from 3.5 to 7.0.

Examples of suitable weak monocarboxylic acids include, but are not limited to, aryl, (hetero)cyclic, alkyl, and/or aliphatic monocarboxylic acids such as, for example, acetic acid, mono, di, or tri chloroacetic acid, glyoxylic acid, glycolic acid, acrylic acid, methacrylic acid, pyruvic acid, propionic acid, D-gluconic acid, and D-galacturonic acid.

A structural example of a monocarboxylic acid suitable for use in the present invention is shown in formula (I) below:

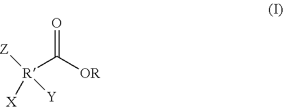

in which:

R denotes H, Li⁺, Na⁺, K⁺, or NH₄⁺;

R′ denotes an alkyl, alkylene, aryl, cyclic, or heterocyclic group containing up to 12 carbon atoms, and which can contain intermittent heteroatoms such as nitrogen and oxygen; and

X, Y and Z, which may be identical or different, denote H, OH, OR (where R is as defined above or denotes CH₃), NH₂, or a halogen atom, or X and Y denote one unique oxygen atom, Z being as defined above.

Aryl and (hetero)cyclic monocarboxylic acids are, in particular, compounds comprising a carboxylic acid moiety on a saturated or unsaturated single or multiple ring containing 5 to 12 carbon atoms which can further contain intermittent nitrogen or oxygen atoms, such as, for example, lactam or lactone. Substituents on the ring(s) may include —H, ═O, —OH, —OR, —NH₂ halides, or combinations thereof.

Hydroxycarboxylic Acid

In one embodiment, hydroxycarboxylic acid is preferable. Hydroxycarboxylic acid having a pKa greater than 3.5 is more preferable. In a particular embodiment, hydroxycarboxylic acids such as glycolic acid, lactic acid, glycerine acid, hydroxyl fatty acid and the like are preferable. Hydroxyl fatty acid includes, for example, 2-Hydroxycaproic acid, 2-Hydroxycaprylic acid, 2-Hydroxylaulic acid, 2-Hydroxymyristic acid, 2-Hydroxypalmitic acid, 2-Hydroxystearic acid, 2-Hydroxyarachidic acid, 2-Hydroxybehenic acid, 2-Hydroxytricosanoic acid, 2-Hydroxylignoceric acid, 2-Hydroxycapric acid, 3-Hydroxypelargonic acid, 3-Hydroxycapric acid, 3-Hydroxyhendacanoic acid, 3-Hydroxylaulic acid, 3-Hydroxytridecylic acid, 3-Hydroxymyristic acid, 3-Hydroxypalmitic acid, 3-Hydroxymargalic acid, 3-Hydroxystearic acid, 3-Hydroxycaprylic acid, 3-Hydroxycaproic acid, 6-Hydroxystearic acid, 10-Hydroxycapric acid, 12-Hydroxystearic acid, 15-Hydroxypentadecylic acid, 16-Hydroxypalmitic acid, 17-Hydroxymargalic acid, 20-Hydroxyarachidic acid, 22-Hydroxybehenic acid.

Dicarboxylic Acid

Suitable weak dicarboxylic acids for use in the present invention are those having a pKa₁ or a pKa2 of equal to or greater than 3.5, preferably from 3.5 to 8.0, and more preferably from 3.5 to 7.0.

Examples of suitable weak dicarboxylic acids include, but are not limited to, aryl, (hetero)cyclic, alkyl, and/or aliphatic dicarboxylic acids.

Suitable representatives thereof include malic acid, maleic acid, itaconic acid, oxalic acid, malonic acid, mesoxalic acid, fumaric acid, succinic acid, tartaric acid, alpha-ketoglutaric acid, iminodiacetic acid, galactartic acid, adipic acid, glutaric acid, their salts, and mixtures thereof.

A structural example of a dicarboxylic acid suitable for use in the present invention is shown in formula (II) below:

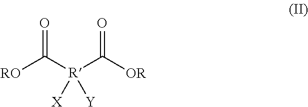

in which

R denotes H, Li⁺, Na⁺, K⁺, or NH₄⁺;

R′ denotes an alkyl, alkylene, aryl, cyclic, or heterocyclic group containing up to 12 carbon atoms, and which can contain intermittent heteroatoms such as nitrogen and oxygen; and

X and Y, which may be identical or different, denote H, OH, OR (where R is as defined above or denotes CH₃), NH₂, or a halogen atom, or X and Y denote one unique oxygen atom.

Aryl and (hetero)cyclic dicarboxylic acids are, in particular, compounds comprising two carboxylic acid moieties on a saturated or unsaturated single or multiple ring containing 5 to 12 carbon atoms which can further contain intermittent nitrogen or oxygen atoms, such as, for example, lactam or lactone. Substituents on the ring(s) may include —H, ═O, —OH, —OR, —NH₂ halides, or combinations thereof.

Particularly preferred weak dicarboxylic acids include dicarboxylic acid having a C₄₋₁₀ carbon chain, such as succinic acid, glutaric acid, adipic acid, pimelic acid, suberic acid, azelaic acid, and sebacic acid. In another particular embodiment, C₃₋₇ cycloalkane-based dicarboxylic acid is preferable, such as 1,2-trans-cyclopropanedicarboxylic acid, 1,3-trans-cyclobutanedicarboxylic acid, 1,2-trans-cyclopentanedicarboxylic acid, 2-trans-cyclohexanedicarboxylic acid, 1,4-trans-cyclohexanedicarboxylic acid, 1,3-trans-cyclohexanedicarboxylicacid, 1,3-trans-cyclopentanedicarboxylic acid, their salts, and mixtures thereof.

Tricarboxylic Acid

Suitable weak tricarboxylic acids for use in the present invention are those having a pKₐ₁, a pKₐ₂, or a pKₐ₃ of equal to or greater than 3.5, preferably from 3.5 to 8.0, and more preferably from 3.5 to 7.0.

Examples of suitable weak tricarboxylic acids include, but are not limited to, aryl, alkyl, or aliphatic tricarboxylic acids such as domoic or nitrilotriacetic acids.

A structural example of a tricarboxylic acid suitable for use in the present invention is shown in figure (III) below:

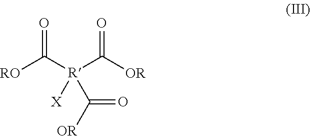

in which R denotes H, Li⁺, Na⁺, K⁺, or NH₄⁺;

R′ denotes an alkyl, alkylene, aryl, cyclic, or heterocyclic group containing up to 12 carbon atoms, and which can contain intermittent heteroatoms such as nitrogen and oxygen; and

X denotes H, OH, OR (where R is as defined above or denotes CH₃), NH₂, or a halogen atom.

Aryl and (hetero)cyclic tricarboxylic acids are, in particular, compounds comprising three carboxylic acid moieties on a saturated or unsaturated single or multiple ring containing 5 to 12 carbon atoms which can further contain intermittent nitrogen or oxygen atoms, such as, for example, lactam or lactone. Substituents on the ring(s) may include —H, ═O, —OH, —OR, —NH₂, halides, or combinations thereof.

Preferred weak acid compounds are not limited, but for example, dicarboxylic acid, and hydroxycarboxylic acid are preferable.

In one embodiment, preferable are carboxylic acids having a pKa value greater than 3.5, which are exemplified as follows, but not limited thereto. The value in the brackets after the species name describes its pKa value:

formic acid (3.77), glycolic acid (3.82), lactic acid (3.86), succinic acid (4.19), benzoic acid (4.21), glutaric acid (4.34), adipic acid (4.42), azelaic acid (4.55), and acetic acid (4.76).

In another embodiment, the compound having a carboxylic acid moiety may be an anionic polymer compound derived from a carboxylic acid, preferably having a molecular weight greater than 1000, preferably higher than 2000.

Anionic Polymer Compound Having at Least One Carboxylic Acid Moiety

In one embodiment, an anionic polymer compound having at least one carboxylic acid moiety containing unit(s) derived from a carboxylic acid is preferable. An anionic polymer compound derived from a carboxylic acid having a pKa greater than 3.5 is more preferable. Anionic polymers are polymers containing units derived from a carboxylic acid, and usually have a molecular weight of 500 to 5,000,000. These polymers are water-soluble polymers, it being possible for this solubility to be obtained by neutralization of the acid groups with an alkali, such as sodium hydroxide, potassium hydroxide, ammonia, or an amine, like mono-, di-, or tri-ethanolamine, 2-amino-2-methylpropanol or 2-amino-2-methylpropane-1,3-diol, mono-, di-, or tri-ethylamine, mono-, di-, or tri-propylamine or isopropylamine.

The carboxylic acid groups can be provided by unsaturated monocarboxylic or dicarboxylic acids, such as those corresponding to the formula:

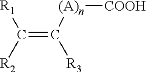

in which n is 0 or an integer from 1 to 10, A denotes a methylene group optionally joined to the carbon atom of the unsaturated group, or to the adjacent methylene group in the case where n is greater than 1, via a hetero-atom, such as oxygen or sulphur, R₁ denotes a hydrogen atom or a phenyl or benzyl group, R₂ denotes a hydrogen atom, a lower alkyl group, or a carboxyl group, and R₃ denotes a hydrogen atom, a lower alkyl group, a group —CH₂—COOH, or a phenyl or benzyl group.

In the above-mentioned formula, a lower alkyl radical preferably is one having 1 to 4 carbon atoms, in particular methyl or ethyl.

According to the present invention, the preferred anionic polymers containing carboxylic acid groups are:

(A) Homopolymers or copolymers of acrylic or methacrylic acid or salts thereof, and, in particular, the products sold under the name VERSICOL E or K by ALLIED COLLOID, under the name ULTRAHOLD 8 by CIBA GEIGY, and under the name DARVAN No. 7 by Van der BILT; acrylic acid/acrylamide copolymers sold in the form of their sodium salt under the name RETEN 421, 423, or 425 by HERCULES; and the sodium salts of polyhydroxycarboxylic acids, sold under the name HYDAGEN F by HENKEL.

(B) Copolymers of acrylic or methacrylic acid with a monoethylenic monomer, such as ethylene, styrene, a vinyl or allyl ester or an acrylic or methacrylic acid ester, optionally grafted onto a polyalkylene glycol, such as polyethylene glycol, and optionally crosslinked. Polymers of this type are described, in particular, in French Pat. No. 1 222 944 and German Specification No. 2 330 956. Other such copolymers contain an optionally N-alkylated and/or N-hydroxylated acrylamide unit in their chain, such as those described, in particular, in Luxembourg Patent Application Nos. 75 370 and 75 371, or those sold under the name QUADRAMER 5 by American Cyanamid.

(C) Copolymers derived from crotonic acid, such as those containing, in their chain, vinyl acetate or propionate units and optionally other monomers such as allyl or methallyl esters, a vinyl ether or a vinyl ester of a saturated linear or branched carboxylic acid with a long (generally containing at least 8 carbon atoms) hydrocarbon chain, such as those containing at least 5 carbon atoms, it being possible, if appropriate, for these polymers to be grafted and crosslinked, or also a vinyl, allyl, or methallyl ester of an alpha- or beta-cyclic carboxylic acid. Polymers of this type are described, inter alia, in French Pat. Nos. 1 222 944, 1 580 545, 2 265 782, 2 265 781, and 1 564 110 and French Specification No. 2 439 798. Commercial products included in this class are the resins 28-29-30, 26-13-14, and 28-13-10 sold by National Starch.

(D) Polymers derived from maleic, fumaric, and itaconic acids or anhydrides with vinyl esters, vinyl ethers, vinyl halides, phenylvinyl derivatives, acrylic acid and its esters; these polymers can be esterified. Polymers of this type are described, in particular, in U.S. Pat. Nos. 2,047,398, 2,723,248, and 2,102,113, and British Pat. No. 839 805. They are in particular copolymers derived from maleic anhydride and vinyl ethers, such as sodium styrene/maleic anhydrate copolymer sold under the name SMA 1000 HNA by Cray Valley, poly(methyl vinyl ether/maleic anhydride) sold under the name GANTREZ AN or ES by General Anilin, or ethylene/maleic anhydride copolymer sold under the name EMA 1325 by MONSANTO. More preferable is sodium styrene/maleic anhydrate copolymer sold under the name SMA 1000 HNA by Cray

Valley. Other polymers included in this class are copolymers of maleic, citraconic, and itaconic anhydrides with an allyl or methallyl ester optionally containing an acrylamido or methacrylamido group, or with an alpha-olefine, acrylic or methacrylic acid ester, acrylic or methacrylic acid, or vinylpyrrolidone unit in their chain; the anhydride groups can be monoesterified or monoamidified; these polymers are described in French Specification Nos. 76/13 929 and 76/20 917.

(E) Polyacrylamides containing carboxylate groups.

Examples of suitable carboxylic acids include, but are not limited to, aryl, (hetero)cyclic, alkyl, and/or aliphatic monocarboxylic acids such as, for example, acetic acid, mono, di, or tri chloroacetic acid, glyoxylic acid, glycolic acid, acrylic acid, methacrylic acid, pyruvic acid, propionic acid, D-gluconic acid, and D-galacturonic acid.

In addition to the above specified carboxylic acids, an anionic polymer compound derived from maleic, fumaric, and itaconic acids or anhydrides are also preferable.

In one embodiment, the amount of the (ii) compound having at least one carboxylic acid moiety may be from 0.2% to 10.0%, preferably from 0.3% to 8.0%, and more preferably from 0.5% to 5.0% by weight in relation to the total weight of the cosmetic composition.

Physiologically Acceptable Medium

Besides the compounds indicated above, the cosmetic composition according to the present invention comprises a physiologically acceptable medium.

The term “physiologically acceptable medium” is intended to denote a medium that is particularly suitable for applying a composition according to the present invention to the skin.

The physiologically acceptable medium is generally adapted to the nature of the support onto which the cosmetic composition is to be applied, and also to the form in which the cosmetic composition is to be packaged.

The cosmetic composition according to the present invention may be a dispersion or an emulsion.

A dispersion may be made as an aqueous phase or as an oily phase. An emulsion may have an oily or aqueous continuous phase. Such an emulsion may be, for example, an inverse (W/O) emulsion or a direct (O/W) emulsion, or alternatively a multiple emulsion (W/O/W or O/W/O).

(c) Aqueous Phase

The cosmetic composition according to the present invention comprises an aqueous phase. The aqueous phase comprises water. The water that is suitable for use in the present invention may be a floral water such as cornflower water and/or a mineral water such as Vittel water, Lucas water or La Roche Posay water, and/or a spring water.

The aqueous phase may also comprise water-miscible organic solvents (at room temperature: 25° C.), for instance monoalcohols containing from 2 to 6 carbon atoms, such as ethanol or isopropanol; polyols especially containing from 2 to 20 carbon atoms, preferably containing from 2 to 10 carbon atoms, and preferentially containing from 2 to 6 carbon atoms, such as glycerol, propylene glycol, butylene glycol, pentylene glycol, hexylene glycol, dipropylene glycol, or diethylene glycol; glycol ethers (especially containing from 3 to 16 carbon atoms) such as mono-, di-, or tripropylene glycol (C₁-C₄)alkyl ethers, mono-, di-, or friethylene glycol (C₁-C₄)alkyl ethers, and mixtures thereof.

The aqueous phase may also comprise stabilizers, for example sodium chloride, magnesium dichloride or magnesium sulfate.

The aqueous phase may also comprise any water-soluble or water-dispersible compound that is compatible with an aqueous phase, such as gelling agents, film-forming polymers, thickeners, or surfactants, and mixtures thereof.

In particular, the cosmetic composition according to the present invention may comprise an aqueous phase in a content ranging from 1% to 80% by weight, especially from 5% to 50%, and more particularly from 10% to 45% by weight in relation to the total weight of the cosmetic composition.

Fatty Phase

The cosmetic composition according to the present invention may comprise at least one liquid and/or solid fatty phase.

According to one embodiment, the cosmetic composition according to the present invention is in the form of an emulsion.

In particular, the cosmetic composition according to the present invention may comprise at least one liquid fatty phase, especially at least one oil as mentioned below.

The term “oil” means any fatty substance that is in liquid form at room temperature (20-25° C.) and at atmospheric pressure.

The composition of the present invention may comprise a liquid fatty phase in a content ranging from 1% to 90%, preferably from 5% to 80%, more preferably from 10% to 70%, and still more preferably from 20% to 50% by weight in relation to the total weight of the cosmetic composition.

The oily phase that is suitable for preparing the cosmetic compositions according to the present invention may comprise hydrocarbon-based oils, silicone oils, fluoro oils, or non-fluoro oils, or mixtures thereof.

The oils may be volatile or non-volatile. The oils may be of animal, plant, mineral, or synthetic origin. The term “non-volatile oil” means an oil that remains on the skin or the keratin fiber at room temperature and atmospheric pressure. More specifically, a non-volatile oil has an evaporation rate strictly less than 0.01 mg/cm²/min.

To measure this evaporation rate, 15 g of an oil or of an oil mixture to be tested are placed in a crystallizing dish 7 cm in diameter, which is placed on a balance in a large chamber of about 0.3 m³ that is temperature-regulated, at a temperature of 25° C., and hygrometry-regulated, at a relative humidity of 50%. The liquid is allowed to evaporate freely, without stirring it, while providing ventilation by means of a fan (Papst-Motoren, reference 8550 N, rotating at 2700 rpm) placed in a vertical position above the crystallizing dish containing said oil or said mixture, the blades being directed towards the crystallizing dish, 20 cm away from the bottom of the crystallizing dish. The mass of oil remaining in the crystallizing dish is measured at regular intervals. The evaporation rates are expressed in mg of oil evaporated per unit of area (cm²) and per unit of time (minutes).

The term “volatile oil” means any non-aqueous medium that is capable of evaporating on contact with the skin or the lips in less than one hour, at room temperature and atmospheric pressure. The volatile oil is a cosmetic volatile oil, which is liquid at room temperature. More specifically, a volatile oil has an evaporation rate of between 0.01 and 200 mg/cm²/min, limits included.

For the purposes of the present invention, the term “silicone oil” means an oil comprising at least one silicon atom, and especially at least one Si—O group.

The term “fluoro oil” means an oil comprising at least one fluorine atom.

The term “hydrocarbon-based oil” means an oil mainly containing hydrogen and carbon atoms.

The oils may optionally comprise oxygen, nitrogen, sulfur, and/or phosphorus atoms, for example in the form of hydroxyl or acid radicals.

Volatile Oils

The volatile oils may be chosen from hydrocarbon-based oils containing from 8 to 16 carbon atoms, and especially C₈-C₁₆ branched alkanes (also known as isoparaffins), for instance isododecane (also known as 2,2,4,4,6-pentamethylheptane), isodecane, and isohexadecane, for instance the oils sold under the trade names Isopar® or Permethyl®.

Volatile oils that may also be used include volatile silicones, for instance volatile linear or cyclic silicone oils, especially those with a viscosity of less than or equal to 8 centistokes (cSt) (8×10⁻⁶ m²/s), and especially containing from 2 to 10 silicon atoms and in particular from 2 to 7 silicon atoms, these silicones optionally comprising alkyl or alkoxy groups containing from 1 to 10 carbon atoms. As volatile silicone oils that may be used in the present invention, mention may be made especially of dimethicones with viscosities of 5 and 6 cSt, octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane, and dodecamethylpentasiloxane, and mixtures thereof.

According to one embodiment, the cosmetic composition according to the present invention may comprise from 1% to 80% by weight, or even from 5% to 70% by weight, or even from 10% to 60% by weight, and especially from 15% to 50% by weight of volatile oil relative to the total weight of the cosmetic composition.

Non-Volatile Oils

The non-volatile oils may be chosen especially from non-volatile hydrocarbon-based, fluoro and/or silicone oils.

Non-volatile hydrocarbon-based oils that may especially be mentioned include:
hydrocarbon-based oils of animal origin, such as perhydrosqualene,
hydrocarbon-based oils of plant origin, such as phytostearyl esters, such as phytostearyl oleate, phytostearyl isostearate, and lauroyl/octyldodecyl/phytostearyl glutamate (Ajinomoto, Eldew PS203), triglycerides formed from fatty acid esters of glycerol, in particular in which the fatty acids may have chain lengths ranging from C₄ to C₃₆, and especially from C₁₈ to C₃₆, these oils possibly being linear or branched, and saturated or unsaturated; these oils may especially be heptanoic or octanoic triglycerides, shea oil, alfalfa oil, poppy oil, winter squash oil, millet oil, barley oil, quinoa oil, rye oil, candlenut oil, passionflower oil, shea butter, aloe vera oil, sweet almond oil, peach stone oil, groundnut oil, argan oil, avocado oil, baobab oil, borage oil, broccoli oil, calendula oil, camelina oil, canola oil, carrot oil, safflower oil, flax oil, rapeseed oil, cotton oil, coconut oil, marrow seed oil, wheatgerm oil, jojoba oil, lily oil, macadamia oil, corn oil, meadowfoam oil, St John's Wort oil, monoi oil, hazelnut oil, apricot kernel oil, walnut oil, olive oil, evening primrose oil, palm oil, blackcurrant pip oil, kiwi seed oil, grapeseed oil, pistachio oil, winter squash oil, pumpkin oil, musk rose oil, sesame oil, soybean oil, sunflower oil, castor oil, and watermelon seed oil, and mixtures thereof, or alternatively caprylic/capric acid triglycerides, such as those sold by the company Stearineries Dubois or those sold under the names Miglyol 810®, 812®, and 818® by the company Dynamit Nobel,
linear or branched hydrocarbons of mineral or synthetic origin, such as liquid paraffins and derivatives thereof, petroleum jelly, polydecenes, polybutenes, hydrogenated polyisobutene such as Parleam, and squalane;
synthetic ethers containing from 10 to 40 carbon atoms;
synthetic esters, for instance the oils of formula R₁COOR₂, in which R₁ represents a linear or branched fatty acid residue containing from 1 to 40 carbon atoms and R₂ represents a hydrocarbon-based chain, which is especially branched, containing from 1 to 40 carbon atoms, on condition that the sum of the number of carbon atoms in the chains R₁ and R₂ is greater than or equal to 10. The esters may be chosen especially from fatty acid esters of alcohols, for instance cetostearyl octanoate, isopropyl alcohol esters, such as isopropyl myristate, isopropyl palmitate, ethyl palmitate, 2-ethylhexyl palmitate, isopropyl stearate, isopropyl isostearate, isostearyl isostearate, and octyl stearate, hydroxylated esters, for instance isostearyl lactate, octyl hydroxystearate, diisopropyl adipate, heptanoates, and especially isostearyl heptanoate, alcohol or polyalcohol octanoates, decanoates, or ricinoleates, for instance propylene glycol dioctanoate, cetyl octanoate, tridecyl octanoate, 2-ethylhexyl 4-diheptanoate, and 2-ethylhexyl palmitate, alkyl benzoates, polyethylene glycol diheptanoate, propylene glycol 2-diethylhexanoate, and mixtures thereof, C₁₂-C₁₅ alcohol benzoates, hexyl laurate, neopentanoic acid esters, for instance isodecyl neopentanoate, isotridecyl neopentanoate, isostearyl neopentanoate, and octyldodecyl neopentanoate, isononanoic acid esters, for instance isononyl isononanoate, isotridecyl isononanoate, and octyl isononanoate, hydroxylated esters, for instance isostearyl lactate and diisostearyl malate,
polyol esters and pentaerythritol esters, for instance dipentaerythrityl tetrahydroxystearate/tetraisostearate,
esters of diol dimers and of diacid dimers, such as Lusplan DD-DA5® and Lusplan DD-DA7® sold by the company Nippon Fine Chemical and described in patent application US 2004-175 338,
copolymers of a diol dimer and of a diacid dimer and esters thereof, such as dilinoleyl diol dimer/dilinoleic dimer copolymers, and esters thereof, for instance Plandool-G,
copolymers of polyols and of diacid dimers, and esters thereof, such as Hailuscent ISDA, or dilinoleic acid/butanediol copolymers,
fatty alcohols that are liquid at room temperature, with a branched and/or unsaturated carbon-based chain containing from 12 to 26 carbon atoms, for instance 2-octyldodecanol, isostearyl alcohol, oleyl alcohol, 2-hexyldecanol, 2-butyloctanol, and 2-undecylpentadecanol;
C₁₂-C₂₂ higher fatty acids, such as oleic acid, linoleic acid, or linolenic acid, and mixtures thereof,
dialkyl carbonates, the two alkyl chains possibly being identical or different, such as dicaprylyl carbonate sold under the name Cetiol CC® by Cognis,
oils of high molar mass, in particular having a molar mass ranging from about 400 to about 10,000 g/mol, in particular from about 650 to about 10,000 g/mol, more particularly from about 750 to about 7500 g/mol, and even more particularly ranging from about 1000 to about 5000 g/mol. As oils of high molar mass that may be used in the present invention, mention may especially be made of oils chosen from:
   lipophilic polymers,
   linear fatty acid esters with a total carbon number ranging from 35 to 70,
   hydroxylated esters,
   aromatic esters,
   C₂₄-C₂₈ branched fatty acid or fatty alcohol esters,
   silicone oils,
   oils of plant origin, and
   mixtures thereof;
optionally partially hydrocarbon-based and/or silicone fluoro oils, for instance fluorosilicone oils, fluoropolyethers, and fluorosilicones as described in document EP-A-847 752;
silicone oils, for instance linear or cyclic non-volatile polydimethylsiloxanes (PDMS); polydimethylsiloxanes comprising alkyl, alkoxy, or phenyl groups, which are pendant or at the end of a silicone chain, these groups containing from 2 to 24 carbon atoms; phenyl silicones, for instance phenyl trimethicones, phenyl dimethicones, phenyl trimethylsiloxy diphenyl siloxanes, diphenyl dimethicones, diphenyl methyldiphenyl trisiloxanes, and 2-phenylethyl trimethylsiloxy silicates, and
mixtures thereof.

According to one particular embodiment, the fatty phase of the cosmetic composition according to the present invention may contain only volatile compounds.

Dyestuffs

The cosmetic composition according to the present invention may also comprise at least one dyestuff.

The amount of dyestuff(s) in the cosmetic composition according to the present invention will generally range from 0 to 25%, preferably from 2 to 15%, and more preferably from 5 to 15% by weight of the total weight of the cosmetic composition.

The cosmetic composition according to the present invention may incorporate at least one dyestuff chosen from mineral or organic pigments conventionally used in cosmetic compositions, liposoluble or water-soluble dyes, materials with a specific optical effect, and mixtures thereof.

The term “pigments” should be understood to mean white or colored, inorganic or organic particles which are insoluble in an aqueous solution and are intended for coloring and/or opacifying the resulting film.

As inorganic pigments that can be used in the present invention, mention may be made of titanium oxides, zirconium oxides, or cerium oxides, and also zinc oxides, iron oxides, or chromium oxides, ferric blue, manganese violet, ultramarine blue, and chromium hydrate. According to one particular mode of the present invention, the mineral pigments will be chosen from iron oxides and titanium oxides, and mixtures thereof.

It may also be a pigment having a structure that may be, for example, of sericite/brown iron oxide/titanium dioxide/silica type. Such a pigment is sold, for example, under the reference Coverleaf NS or JS by the company Chemicals and Catalysts, and has a contrast ratio in the region of 30.

The colorant may also comprise a pigment having a structure which may be, for example, of the type such as silica microspheres containing iron oxide. An example of a pigment having this structure is the product sold by the company Miyoshi under the reference PC Ball PC-LL-100 P, this pigment being constituted of silica microspheres containing yellow iron oxide.

Among the organic pigments that may be used in the present invention, mention may be made of carbon black, pigments of D&C type, lakes based on cochineal carmine or on barium, strontium, calcium, or aluminium, or alternatively the diketopyrrolopyrroles (DPP) described in documents EP 0 542 669, EP 0 787 730, EP 0 787 731, and WO 96/08537.

The cosmetic composition according to the present invention may also comprise water-soluble or fat-soluble dyes. The liposoluble dyes are, for example, Sudan Red, DC Red 17, DC Green 6, β-carotene, soybean oil, Sudan Brown, DC Yellow 11, DC Violet 2, DC Orange 5, and quinoline yellow. The water-soluble dyes are, for example, beetroot juice and caramel.

Additional Fillers

The cosmetic composition according to the present invention may also comprise at least one additional filler, of organic or mineral nature, making it possible especially to give it additional matte-effect or covering properties, and/or improved stability with regard to exudation and migration-resistance properties after application.

The term “filler” should be understood to mean colorless or white solid particles of any shape which are in a form that is insoluble and dispersed in the medium of the cosmetic composition. These particles, of mineral or organic nature, can give body or rigidity to the cosmetic composition and/or softness and uniformity to the makeup.

The fillers used in the cosmetic compositions according to the present invention may be in lamellar, globular, or spherical form, in the form of fibers, or in any other intermediate form between these defined forms.

The fillers according to the present invention may or may not be surface-coated, and in particular they may be surface-treated with silicones, amino acids, fluoro derivatives, or any other substance that promotes the dispersion and compatibility of the filler in the cosmetic composition.

Examples of mineral fillers that may be mentioned include talc, mica, silica, kaolin, calcium carbonate, magnesium carbonate, hydroxyapatite, glass, or ceramic microcapsules.

Examples of organic fillers that may be mentioned include polyethylene powder or polymethyl methacrylate powder, polytetrafluoroethylene (Teflon) powders, lauroyl lysine, hexamethylene diisocyanate/trimethylol hexyl lactone copolymer powder (Plastic Powder from Toshiki), silicone resin microbeads (for example Tospearl from Toshiba), natural or synthetic micronized waxes, metal soaps derived from organic carboxylic acids containing from 8 to 22 carbon atoms and preferably from 12 to 18 carbon atoms, for example zinc stearate, magnesium stearate, lithium stearate, zinc laurate, or magnesium myristate, and polyurethane powders, in particular crosslinked polyurethane powders comprising a copolymer, the said copolymer comprising trimethylol hexyl lactone. It may in particular be a hexamethylene diisocyanate/trimethylol hexyl lactone polymer. Such particles are especially commercially available, for example, under the name Plastic Powder D-400® or Plastic Powder D-800® from the company Toshiki, and mixtures thereof.

The amount of filler(s) in the cosmetic composition of the present invention will generally range from 0 to 25%, preferably from 2 to 15%, and more preferably from 5 to 15% by weight of the total weight of the cosmetic composition.

Additives

In a particular embodiment, the cosmetic composition according to the present invention further comprises at least one compound chosen from water, hydrophilic solvents, lipophilic solvents, oils, and mixtures thereof.

The cosmetic composition according to the present invention may also comprise any additive usually used in the field under consideration, chosen, for example, from gums, anionic, cationic, amphoteric, or nonionic surfactants, silicone surfactants, resins, thickening agents, structuring agents such as waxes, dispersants, antioxidants, essential oils, preserving agents, fragrances, neutralizers, antiseptics, UV-screening agents, cosmetic active agents, such as vitamins, moisturizers, emollients, or collagen-protecting agents, and mixtures thereof.

It is a matter of routine operations for a person skilled in the art to adjust the nature and amount of the additives present in the cosmetic compositions in accordance with the present invention such that the desired cosmetic properties and stability properties thereof are not thereby affected.

The cosmetic composition according to the present invention may be in the form of a skin makeup product, in particular a foundation, a hot-cast foundation product, a body makeup product, a concealer, an eyeshadow, a lipstick, or a body deodorant. The cosmetic composition may be in the form of a gel, in the form of cream, in the form of a stick or wand, or in the form of a soft paste. In a particular embodiment, the cosmetic composition may be a liquid foundation.

A care composition according to the present invention may in particular be an anti-sun composition. Preferably, the cosmetic composition according to the present invention is in the form of a fluid primer or a fluid foundation.

In one embodiment, the cosmetic composition may be in the form of an emulsion or in the form of a clear lotion.

In a particular embodiment, the present invention relates to use of a combination of (i) at least one oxide of an alkaline earth metal and (ii) at least one compound having at least one carboxylic acid moiety as an agent for a long-wear makeup effect.

[Cosmetic Process]

The present invention also relates to a cosmetic process comprising a step of applying the cosmetic composition according to the present invention, on the skin, in particular the face. In a particular embodiment, the cosmetic composition is applied alone or as a base or primer under a skin care product or a makeup product. The cosmetic process preferably includes making up and/or caring for the skin, preferably facial skin.

The cosmetic composition used in the cosmetic process according to the present invention is preferably of the leave-in type. The term “leave-in” means a composition that is not intended to be washed out or removed immediately after application.

The cosmetic process according to the present invention can provide sebum solidification or gelification effects, as well as a long-wear effect without causing shine. Thus, the cosmetic process according to the present invention can provide a long wear effect on the skin over time even under hot and/or humid conditions, for example, during the summer.

### EXAMPLES

The present invention will be described in a more detailed manner by way of examples. However, these examples should not be construed as limiting the scope of the present invention.

### TEST EXAMPLES

[Preparation]

The following compositions were prepared by mixing the components shown in Table 1. The numerical values for the amounts of the components are all based on “% by weight” as active raw materials.

Sebum Gelification Test

[Protocol]

The gelification speed of artificial sebum was measured by using the following combination. The mixtures below were each stirred at room temperature for 10 min by a magnetic stirrer. When the magnetic stirrer was stopped due to sebum gelification (solidification) or the composition was moved to the flask wall by the magnetic stirrer and was not returned to the magnetic stirrer, this moment was determined to be the gelification time. One hour after the start of stirring, the cosmetic composition was applied over a contrast card with a 30 μm applicator. The surface shine was then measured at a 60° gloss value of a Glossmeter.

TABLE 1
Composition and results of sebum gelification test

| Test Example No. | 1 | 2 | 3 | 4 | 5 | 6 | 7 | 8 | 9 | 10 | 11 | 12 |
|---|---|---|---|---|---|---|---|---|---|---|---|---|
| Magnesium oxide | 0.1 | 0.1 | 0.1 | 0.1 | 0.1 | 0.1 | 0.1 | 0.1 | 0.1 | — | — | — |
| Zinc oxide | — | — | — | — | — | — | — | — | — | 0.1 | 0.1 | 0.1 |
| Lactic acid 90% | 0.11 | — | — | — | — | — | — | — | — | — | — | — |
| Sodium styrene/maleic | — | 0.1 | — | — | — | — | — | — | — | — | — | — |
| anhydride copolymer | | | | | | | | | | | | |
| (SMA1000HNA: CrayValley) | | | | | | | | | | | | |
| Azelaic acid | — | — | 0.1 | — | — | — | — | — | — | — | — | — |
| Glutamic acid | — | — | — | 0.1 | — | — | — | 0.1 | — | — | — | — |
| Citric acid | — | — | — | — | 0.1 | — | — | — | 0.1 | — | — | 0.1 |
| Water | 0.14 | 0.15 | 0.15 | 0.15 | 0.15 | — | 0.25 | — | — | — | 0.25 | 0.15 |
| Artificial Sebum | 2 | 2 | 2 | 2 | 2 | 2 | 2 | 2 | 2 | 2 | 2 | 2 |
| (see Table 2) | | | | | | | | | | | | |
| Gelification time | 5 | 10 | 10 | 10 | 10 | No | No | No | No | No | 60 | No |
| (min) | | | | | | gel | gel | gel | gel | gel | | gel |
| 60° Gloss | 5 | 5.1 | 17.0 | 5.6 | 4.7 | 83.5 | 86.1 | 77.1 | 83.0 | 79.7 | 13.9 | 74.0 |

### Test Example 1

magnesium oxide, lactic acid, and water

### Test Example 2

magnesium oxide, sodium styrene/maleic anhydride copolymer, and water

### Test Example 3

magnesium oxide, azelaic acid, and water

### Test Example 4

magnesium oxide, glutamic acid, and water

### Test Example 5

magnesium oxide, citric acid, and water

### Test Example 6

magnesium oxide, without acid and without water

### Test Example 7

magnesium oxide and water, without acid

### Test Example 8

magnesium oxide and glutamic acid, without water

### Test Example 9

magnesium oxide and citric acid, without water

### Test Example 10

zinc oxide, without acid and without water

### Test Example 11

zinc oxide and water, without acid

### Test Example 12

zinc oxide, citric acid, and water

Table 2 shows the composition of the artificial sebum used.

TABLE 2
Composition of artificial sebum

| Ingredient | Wt % |
|---|---|
| Triisostearin | 28.7 |
| Parleam | 13.7 |
| Oleic acid | 28.0 |
| Oleyl erucate | 22.9 |
| Octyldodecanol | 6.7 |
| Total | 100 |

[Results]

Test Examples 1 to 5 (containing magnesium oxide, acid, and water) showed a gelification effect of an artificial sebum. Each of the mixtures of Test Examples 1 to 5 showed a lower gloss value. In contrast, sebum solidification effect was not seen in Test Example 6 (magnesium oxide alone), Test Example 7 (magnesium oxide and water, without acid), Test Examples 8 to 9 (magnesium oxide and acid, without water). Test Example 10 (zinc oxide without acid and without water) and Test Example 12 (zinc oxide, acid, and water) did not show a sebum solidification effect. Test Example 11 (zinc oxide and water, without acid) required 60 minutes to show a sebum solidification effect. Test Examples 6 to 12 showed a high value of gloss.

Consequently, it was confirmed that a combination of magnesium oxide, carboxylic acid, and water proved gelification of artificial sebum and reduced shine of artificial sebum.

### FORMULATION EXAMPLES

### Formulation Examples 1 to 3 and 1′-4′ and Comparative Formulation Examples 1 to 4 and 1′-4′

### Foundation

Based on the above experiment, the effect was evaluated in a foundation formula.

[Preparation]

In Table 3 and Table 3′ below, all compositions are expressed in % by weight. They were prepared according to the same protocol: mix oil phase ingredients together and heat up to 60-80° C. until wax was melted, the dispersed pigments and fillers inside the oil phase then proceed to emulsification by adding water phase ingredients.

TABLE 3
Liquid foundation formula

| | Example | | | Comparative Example | | | |
|---|---|---|---|---|---|---|---|
| Formulation Example | 1 | 2 | 3 | 1 | 2 | 3 | 4 |
| PEG-10 dimethicone | 2.5 | 2.5 | 2.5 | 2.5 | 2.5 | 2.5 | 2.5 |
| (KF6017: Shinetsu) | | | | | | | |
| Dimethicone | 14.1 | 13.1 | 13.1 | 15.1 | 14.1 | 13.1 | 15.1 |
| (KF96L 2CS: Shinetsu) | | | | | | | |
| Ethylhexyl Methoxycinnamate | 3 | 3 | 3 | 3 | 3 | 3 | 3 |
| Talc | 0.5 | 0.5 | 0.5 | 0.5 | 0.5 | 0.5 | 0.5 |
| Iron oxide | 11.7 | 11.7 | 11.7 | 11.7 | 11.7 | 11.7 | 11.7 |
| Water | qs 100 | qs 100 | qs 100 | qs 100 | qs 100 | qs 100 | qs 100 |
| Glycerin | 3.5 | 3.5 | 3.5 | 3.5 | 3.5 | 3.5 | 3.5 |
| Alcohol denat. | 8 | 8 | 8 | 8 | 8 | 8 | 8 |
| Butylene glycol | 3 | 3 | 3 | 3 | 3 | 3 | 3 |
| Magnesium oxide | 1.5 | 1.5 | 1.5 | — | 1.5 | 1.5 | 1.5 |
| (Magnesium oxide light: | | | | | | | |
| Dr. Paul Lohmann) | | | | | | | |
| Lactic acid (90%) | 5.56 | — | — | — | — | — | — |
| Sodium styrene/maleic | — | 12.5 | — | — | — | — | — |
| anhydrate copolymer (40%) | | | | | | | |
| (SMA1000HNA: CrayValley) | | | | | | | |
| Azelaic acid (pKa1: 4.55) | — | — | 5 | — | — | — | — |
| Citric acid (pKa1: 2.87) | — | — | — | — | 5 | — | — |
| Glutamic acid (pKa1: 2.2) | — | — | — | — | — | 5 | — |
| Total | 100 | 100 | 100 | 100 | 100 | 100 | 100 |
| (A) With artificial sebum | 41.01 | 37.07 | 36.2 | 191.5 | 68.9 | 105.8 | 150.0 |
| (B) Without artificial sebum | 32.63 | 31.6 | 31.3 | 46.5 | 31.34 | 31.87 | 30.2 |
| Difference (A) − (B) | 8.38 | 5.47 | 4.9 | 145.0 | 37.56 | 73.93 | 119.8 |

TABLE 3′
Liquid foundation formula

| | Example | | | | Comparative Example | | | |
|---|---|---|---|---|---|---|---|---|
| Formulation Example | 1′ | 2′ | 3′ | 4′ | 1′ | 2′ | 3′ | 4′ |
| PEG-10 dimethicone | 2.5 | 2.5 | 2.5 | 2.5 | 2.5 | 2.5 | 2.5 | 2.5 |
| (KF6017: Shinetsu) | | | | | | | | |
| Dimethicone | 14.1 | 13.1 | 13.1 | 13.1 | 15.1 | 14.1 | 13.1 | 15.1 |
| (KF96L 2CS: Shinetsu) | | | | | | | | |
| Ethylhexyl Methoxycinnamate | 3 | 3 | 3 | 3 | 3 | 3 | 3 | 3 |
| Talc | 0.5 | 0.5 | 0.5 | 0.5 | 0.5 | 0.5 | 0.5 | 0.5 |
| Pigment (TiO₂ + Iron oxide) | 11.7 | 11.7 | 11.7 | 11.7 | 11.7 | 11.7 | 11.7 | 11.7 |
| Water | qs 100 | qs 100 | qs 100 | qs 100 | qs 100 | qs 100 | qs 100 | qs 100 |
| Glycerin | 3.5 | 3.5 | 3.5 | 3.5 | 3.5 | 3.5 | 3.5 | 3.5 |
| Alcohol denat. | 8 | 8 | 8 | 8 | 8 | 8 | 8 | 8 |
| Butylene glycol | 3 | 3 | 3 | 3 | 3 | 3 | 3 | 3 |
| Magnesium oxide | 1.5 | 1.5 | 1.5 | 0.5 | — | 1.5 | 1.5 | 1.5 |
| (Magnesium oxide light: | | | | | | | | |
| Dr. Paul Lohmann) | | | | | | | | |
| Lactic acid (90%) | 5.56 | — | — | — | — | — | — | — |
| Sodium styrene/maleic | — | 12.5 | — | — | — | — | — | — |
| anhydrate copolymer (40%) | | | | | | | | |
| (SMA1000HNA: CrayValley) | | | | | | | | |
| Azelaic acid (pKa1: 4.55) | — | — | 5 | — | — | — | — | — |
| Citric acid (pKa1: 2.87) | — | — | — | — | — | 5 | — | — |
| Glutamic acid (pKa1: 2.2) | — | — | — | — | — | — | 5 | — |
| 12-Hydroxystearic acid | — | — | — | 4 | — | — | — | — |
| (pKa: 4.75) | | | | | | | | |
| Total | 100 | 100 | 100 | 100 | 100 | 100 | 100 | 100 |
| (A) With artificial sebum | 41.01 | 37.07 | 36.2 | 24.91 | 191.5 | 68.9 | 105.8 | 150.0 |
| (B) Without artificial sebum | 32.63 | 31.6 | 31.3 | 23.14 | 46.5 | 31.34 | 31.87 | 30.2 |
| Difference (A) − (B) | 8.38 | 5.47 | 4.9 | 1.8 | 145.0 | 37.56 | 73.93 | 119.8 |

Sebum Resistance Test of Liquid Foundation

[Protocol]

Each of the liquid foundations according to Formulation Examples 1-3 or Comparative Formulation Examples 1-4) (1.0 g) and artificial sebum (0.2 g) were mixed, and the mixture was applied on artificial leather (SUPPLALE® from. Idemitsu) at 2.4 mg/cm². The artificial leather was then dried at 35° C. for 30 min. The reflectance of the foundation film with artificial sebum was measured by a goniophotometer (A). The reflectance of the foundation film without artificial sebum was also measured by a goniophotometer (B).

[Results]

The formula containing magnesium oxide, carboxylic acid, and water (Formulation Examples 1 to 3) showed a lower reflectance compared with the basic formula (Comparative Formulation Example 1). However, it was shown that the combination of magnesium oxide and a certain type of acid (glutamic acid or citric acid) was not effective (Comparative Formulation Examples 2 and 3). It was also confirmed that magnesium oxide alone in the formula was not effective (Comparative Example 4). A lower reflectance means that the cosmetic effect is less deteriorated. Therefore, this indicates that the formula according to the present invention showed a higher resistance against sebum.

Sebum Resistance Test (Heat Resistance)

[Protocol]

Formula Examples 1 and 2, and Comparative Formula Example 1 were subjected to a sebum resistance test. A liquid foundation and artificial sebum were mixed at a weight ratio of 1 g:0.2 g. The mixture was applied on artificial leather (SUPPLALE® from Idemitsu) at 2.4 mg/cm². The artificial leather was then dried at 31° C., 36° C., or 40° C. respectively for 30 min. The reflectance of the foundation film with artificial sebum was measured by a goniophotometer (A). The reflectance of the foundation without artificial sebum was also measured by a goniophotometer (B).

TABLE 4
Carboxylic acid vs. in vitro wear test results for different temperatures

| | Ex. 1 | | | Ex. 2 | | | Comp. Ex. 4 | | |
|---|---|---|---|---|---|---|---|---|---|
| Formulation Example | (MgO + lactic acid) | | | (MgO + SMA) | | | (MgO alone) | | |
| pKa | 3.86 | | | 3.75-4.0* | | | — | | |
| Drying temperature (° C.) | 31 | 36 | 40 | 31 | 36 | 40 | 31 | 36 | 40 |
| With artificial sebum (A) | 116.7 | 64.9 | 44.4 | 37.2 | 39.8 | 36.1 | 143.8 | 156.3 | 185.4 |
| Without artificial sebum (B) | 37.1 | 31.1 | 30.7 | 24.8 | 26.4 | 28.3 | 40.6 | 40.1 | 41.6 |
| (B) − (A) | 79.6 | 33.8 | 13.7 | 12.4 | 13.4 | 7.8 | 103.2 | 116.2 | 143.8 |

*Referred to WO2006/129127

[Results]

Both lactic acid and sodium styrene/maleic anhydrate copolymer are preferable, and sodium styrene/maleic anhydrate copolymer is more preferable in terms of long-wear effect at a high temperature like 36° C. or 40° C.

### Formulation Examples 4

### Skincare Cream

[Preparation]

In Table 5 below, all compositions are expressed in % by weight. They were prepared according to the same protocol: mix water phase ingredients together and heat up to 60-80° C. until xanthan gum was melted, then proceed to emulsification by adding oil phase ingredients.

TABLE 5
Cream type product formula

| | | | Comp. | Comp. |
|---|---|---|---|---|
| Formulation Example | Ex. 4 | Ex. 5 | Ex. 5 | Ex. 6 |
| Magnesium oxide (Magesium | 2 | 2 | — | 2 |
| oxide light: Dr. Paul Lohmann) | | | | |
| Lactic acid (90%) | 11 | — | — | — |
| Sodium styrene/maleic anhydrate | — | 25 | — | — |
| copolymer (40%) (SMA1000HNA: | | | | |
| CrayValley) | | | | |
| Glycerin | 7 | 7 | 7 | 7 |
| Alcohol denat. | 8 | 8 | 8 | 8 |
| Water | qs. 100 | qs. 100 | qs. 100 | qs. 100 |
| Propylene Glycol | 2 | 2 | 2 | 2 |
| Xanthan Gum | 1 | 1 | 1 | 1 |
| Poly Dimethylsiloxane (10 CST) | 10 | 10 | 10 | 10 |
| PEG-12 Dimethicone | 1 | 1 | 1 | 1 |
| Total | 100 | 100 | 100 | 100 |
| (A) With artificial sebum | 53.1 | 47.1 | 178.7 | 80.3 |
| (B) Without artificial sebum | 18.9 | 21.9 | 62.7 | 20.5 |
| Difference (A) − (B) | 34.2 | 25.2 | 116 | 59.8 |

[Results]

The formula containing MgO and acids (Formulation Examples 4 and 5) showed a lower reflectance than the comparative examples (Formulation Examples 5 and 6).

1.-15. (canceled)

16. A cosmetic composition comprising:
(i) at least one oxide of an alkaline earth metal,
(ii) at least one compound comprising at least one carboxylic acid moiety having a pKa value equal to or greater than about 3.5, and
(iii) an aqueous phase.

17. The cosmetic composition according to claim 16, wherein the weight ratio of the (i) at least one oxide of an alkaline earth metal to the (ii) at least one compound comprising at least one carboxylic acid moiety is equal to or less than about 1.0.

18. The cosmetic composition of claim 16, wherein the (i) at least one oxide of an alkaline earth metal is an oxide of magnesium or calcium.

19. The cosmetic composition of claim 16, wherein the (ii) at least one compound comprising at least one carboxylic acid moiety is chosen from non-polymeric compounds having a molecular weight greater than about 150.

20. The cosmetic composition of claim 19, wherein the non-polymeric compound is chosen from monocarboxylic acids, hydroxycarboxylic acids, dicarboxylic acids, tricarboxylic acids, and mixtures thereof.

21. The cosmetic composition of claim 20, wherein the non-polymeric compound is chosen from formic acid, glycolic acid, lactic acid, succinic acid, benzoic acid, glutaric acid, adipic acid, azelaic acid, and acetic acid.

22. The cosmetic composition of claim 16, wherein the (ii) at least one compound comprising at least one carboxylic acid moiety is chosen from anionic polymer compounds derived from carboxylic acids having a molecular weight greater than about 1000.

23. The cosmetic composition of claim 22, wherein the anionic polymer compound derived from a carboxylic acid is a polymer derived from maleic, fumaric, and itaconic acids or anhydrides with vinyl esters, vinyl ethers, vinyl halides, phenylvinyl derivatives, acrylic acid, and its esters.

24. The cosmetic composition of claim 16, wherein the amount of the (i) at least one oxide of an alkaline earth metal ranges from about 0.2% to about 5.0% by weight in relation to the total weight of the cosmetic composition.

25. The cosmetic composition of claim 16, wherein the amount of the (ii) at least one compound comprising at least one carboxylic acid moiety ranges from about 0.2% to about 10.0% by weight in relation to the total weight of the cosmetic composition.

26. The cosmetic composition of claim 16, wherein the cosmetic composition is in the form of an emulsion, lotion, gel, or cream.

27. The cosmetic composition of claim 16, wherein the cosmetic composition is a liquid foundation.

28. A method for providing a long-wear cosmetic effect comprising:
applying a cosmetic composition to a keratinous material, said cosmetic composition comprising:
(i) at least one oxide of an alkaline earth metal; and
(ii) at least one compound comprising at least one carboxylic acid moiety;
(iii) an aqueous phase.

29. The method of claim 28, wherein the keratinous material comprises skin.
